SEQUENCE LISTING (2 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - DNA, 489 bp, homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctaccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

(19) United States
(12) Patent Application Publication
(10) Pub. No.: US 2008/0279817 A1
(43) Pub. Date: Nov. 13, 2008

(54) Treatment of Autoimmune Diseases and Allograft Rejection with IL-21

(75) Inventor: Kresten Skak, Soborg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, Bagsvaerd (DK)

(21) Appl. No.: 12/052,787

(22) Filed: Mar. 21, 2008

Related U.S. Application Data
(63) Continuation of application No. 11/482,429, filed on Jul. 7, 2006, now abandoned.
Application No. 11/482,429 is a continuation of application No. PCT/DK2005/000015, filed on Jan. 13, 2005.
(60) Provisional application No. 60/541,021, filed on Feb. 2, 2004.

(30) Foreign Application Priority Data
Jan. 15, 2004 (DK) PA 2004 00043

(51) Int. Cl. A61K 38/20; A61K 31/711; A61P 3/10; A61P 37/00

(52) U.S. Cl. 424/85.2; 514/44

(57) ABSTRACT

The invention provides combination treatments with IL-21, an analogue, a derivative or active fragment thereof, an IL-21 mimetic or IL-21 polynucleotide.

### CROSS-REFERENCE TO RELATED PATENT APPLICATIONS

This patent application is a continuation of patent application Ser. No. 11/482,429, filed Jul. 7, 2006, which is a continuation of International Patent Application PCT/DK2005/000015 (published as WO 2005/067956), filed Jan. 13, 2005, which designates the US, and claims the benefit of U.S. Provisional Patent Application 60/541,021, filed Feb. 2, 2004, and Danish Patent Application PA 2004 00043, filed Jan. 15, 2004, the entirety of each of which being hereby incorporated by reference.

### FIELD OF THE PRESENT INVENTION

The present invention relates to management, treatment and prevention of autoimmune diseases or conditions and allograft rejection, by administering IL-21, an analogue, a derivative or active fragment thereof or by in vitro culturing of cells with IL-21 followed by reintroduction of the cells. Further, the invention also relates to the use of IL-21, an analogue, a derivative or active fragment thereof in combination with other pharmaceutical compounds in the above management, treatment and/or prevention.

### BACKGROUND OF THE INVENTION

In both autoimmune diseases and in allograft rejection T cells and/or B cells are central effector cells required for the pathogenesis. Thus, in allograft rejection T cells recognize allo-MHC expressed by the engrafted tissue, leading to T cell-mediated rejection of the graft. In autoimmune diseases either T cells or B cells may recognize self antigens and elicit an immune response against the self tissue expressing these antigens. Recent literature has demonstrated that subsets of dendritic cells (DC) may determine whether the T cells are primed to become effector cells or to become tolerant cells. Factors influencing this are the specific subtype of DC (myeloid or plasmacytoid DC), the presence of cytokines (e.g. IL-4, IL-10, IL-13, TNF-α), growth factors and antagonists of certain cell surface molecules (non-limiting examples are CD40, DEC-205) during differentiation and/or during maturation. Thus, differentiation of bone marrow-derived DC in the presence of GM-CSF+IL-4 will lead to DC capable of inducing effector T cells whereas differentiation of bone marrow-derived DC in the presence of GM-CSF+IL-10 will lead to DC capable of inducing tolerant T cells or even regulatory T cells that can suppress the effector functions of effector T cells leading to reduced immune responses.

Several publications show that DC are able to induce anergic/tolerogenic/regulatory T cells provides they are stimulated properly, that in turn may lead to protective immune responses that can be used to treat autoimmune conditions (Feili-Hariri et al. 1999, Diabetes 48:2300-2308) and GVHD (Sato et al. 2003, Immunity 18:367-379 and Sato et al. 2003, Blood 101:3581-3589).

IL-21, which has also been termed Zalpha11, is a cytokine that was shown to be produced by activated CD4⁺ T lymphocytes after stimulation with anti-CD3 antibody or phorbol ester plus ionomycin (Parrish-Novak et al. 2000, Nature 408:57-63). Recently, it was demonstrated that IL-21 inhibits maturation of a certain DC subset that was incapable of stimulating a T cell response (Brandt et al. 2003, Blood, DOI 10.1182). Thus, IL-21 can be used to differentiate/mature DC to become regulatory DC that are capable of suppressing T cell responses and/or induce regulatory T cell responses in an antigen-specific or disease-specific manner without general compromising the function of the immune system.

The present invention relates to a method for treating, preventing and/or managing of disorders or conditions where lymphocytes are important for the etiology and/or the pathogenesis of said disorders or conditions, including but not limited to autoimmune diseases and host-versus-graft disease, by administration of IL-21, an analogue, a derivative or active fragment thereof, alone or together with other cytokines, growth factors and/or antigens. Administration of IL-21 will induce a regulatory or tolerogenic phenotype of the DC, which will then modify T cell responses to become anergic, regulatory or tolerogenic. By combining IL-21 treatment with additional DC modifying agents and/or T cell modifying or suppressive agents the suppression of the noxious effector functions may be augmented. The co-administration of antigens allows the DC to take up, process and present fragments of these antigens on MHC molecules, thus allowing the priming of antigen-specific responses. This method has the advantage compared to traditionally applied immunosuppression that it can be applied in an antigen-specific manner, thus not compromising the general function of the immune system. Hence, regulatory DC may for example protect against graft-versus-host disease (GVHD) while maintaining a functional graft-versus-leukemia (GVL) response (Sato et al. 2003, Immunity 18:367-379).

The present invention also provides a method to target IL-21 to the DC by conjugating IL-21 to an antibody recognizing DC specific surface molecules. By targeting IL-21 to the DC adverse effects resulting from the binding of IL-21 to other leukocytes or other cells expressing an IL-21R may be minimized. Furthermore, antibodies directed against proteins expressed only by specific subsets of DC may be used to target IL-21 to these subsets.

The present invention also provides a method to culture DC in vitro with IL-21 with or without other cytokines, growth factors and/or antigens to generate regulatory DC that upon reintroduction in vivo has the capability to suppress immune response. This method has the advantage that inappropriate activation of the immune system resulting from IL-21 binding to other leukocytes can be avoided. Furthermore, this method the DC can take up an process antigens of choice that are added to the culture, hereby avoiding modification of DC responses to other antigens. Thus, this method may also be used to treat autoimmune diseases and allograft rejection.

The present invention also provides IL-21, an analogue, a derivative or active fragment thereof in combination with other pharmaceutical compounds in the management, treatment and/or prevention of autoimmune diseases or conditions and allograft rejection.

### SUMMARY OF THE INVENTION

The present invention provides analogues, derivatives or active fragments of IL-21 as medicaments.

The present invention also provides a pharmaceutical composition comprising an analogue, derivative or active fragment of IL-21 together with pharmaceutical acceptable diluents and/or carriers.

The present invention provides the use of an analogue, derivative or active fragment of IL-21 for the manufacture of a medicament for the treatment or prevention of autoimmune diseases and allograft rejection.

The present invention also provides a method of treating or preventing autoimmune diseases and allograft rejection (host-versus-graft disease) by administering to a patient in need thereof an effective therapeutic amount of an analogue, derivative or active fragment of IL-21.

The present invention also provides methods for combining IL-21 therapy with other agents capable of modifying DC responses and T cell responses.

The present invention also provides methods for combining IL-21 therapy with other agents, such as cytokines, growth factors and/or antigens, involved in autoimmune diseases.

The present invention also provides methods for combining IL-21 therapy with other agents, such as cytokines, growth factors and/or antigens, involved in allograft rejection.

The present invention also relates to conjugating IL-21 to a DC targeting compound, preferentially an antibody or a fragment thereof, in order to direct the therapy against DC.

The present invention also relates to culturing DC with IL-21 together with other agents capable of modifying DC responses and/or together with antigens to induce a regulatory or tolerogenic phenotype of the DC that can be used to treat autoimmune diseases or prevent allograft rejection upon reintroduction in vivo.

### DEFINITIONS

A “polypeptide” is a polymer of amino acid residues linked by peptide bonds, and may be produced naturally or synthetically. Polypeptides of less than about 10 amino acid residues are commonly referred to as “peptides”.

A “protein” is a macromolecule comprising one or more polypeptide chains, which may be produced naturally or synthetically. A protein may also comprise non-peptidic components, such as carbohydrate groups or other non-peptidic substituents. Carbohydrates and other non-peptidic substituents may be added to a protein by the cell in which the protein is produced, and will vary with the type of cell. Carbohydrates and other non-peptidic substituents may also be added synthetically after the cell-based production of the protein. Proteins are defined herein in terms of their amino acid backbone structures; substituents such as carbohydrate groups or other non-peptidic substituents are generally not specified, but may be present nonetheless.

“IL-21” is defined as in International Patent Application No. PCT/US06067, publication no. WO 00/53761, published Sep. 14, 2000, which is hereby incorporated in this application in its entirety. WO 00/53761 discloses IL-21 (as “cytokine zalpha11 ligand”) as SEQ ID No. 2, which is hereby incorporated in this application in its entirety, and which is also shown as SEQ ID No. 2 in this application, as well as methods for producing it and antibodies thereto and a polynucleotide sequence encoding IL-21 as SEQ ID No. 1.

The invention also embraces DNA sequences encoding the peptide as SEQ ID No. 1, functional derivatives and fragments thereof. The present application also describes analogues of IL-21 and derivatives thereof. In the context of the present invention the term “IL-21” thus means IL-21 as described in WO00/53761, while “IL-21 and derivatives thereof” covers as well variants, analogues, derivatives and active fragments thereof, accordingly.

The term “IL-21” is also used to cover IL-21 polypeptides which as used herein should be taken to mean polypeptides with a sequence identity to the polypeptide of SEQ ID No: 2 or their orthologs comprising at least 70%, at least 80%, at least 90%, at least 95%, or greater than 95%. The present invention also includes the use of polypeptides that comprise an amino acid sequence having at least 70%, at least 80%, at least 90%, at least 95% or greater than 95% sequence identity to the sequence of amino acid residues 1 to 162, residues 30 to 162, or residues 33 to 162 of SEQ ID No: 2. Methods for determining percent identity are described below. The present invention also includes the use of IL-21 polypeptides that are part of a fusion protein or chimeric protein.

The term “IL-21 mimetic” as used herein cover a compound which is not an IL-21 polypeptide as described above, but which has the biological activity of IL-21. An IL-21 mimetic may be a peptide, such as a polypeptide or an oligopeptide or may be non-proteins, such as a smaller organic molecule.

The term “IL-21 polynucleotide” as used herein cover a polynucleotide encoding IL-21 or a vector comprising an IL-21 polypeptide or a fragment thereof that have a sequence identity to the entire polypeptide, amino acid residues 1 to 162, residues 30 to 162, or residues 33 to 162 of SEQ ID No: 2, or their orthologs, of at least 70%, at least 80%, at least 90%, at least 95%, or greater than 95% sequence identity. An example of such a polynucleotide is shown as SEQ ID No. 1 coding for a polypeptide with a sequence as shown in SEQ ID No. 2.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

The term “autoimmune diseases” as used herein cover all conditions in which the body recognizes its own tissues as foreign and directs an immune response against them. Condition that fall under the term autoimmune diseases include, but are not limited to rheumatoid arthritis (RA), multiple sclerosis (MS), systemic lupus erythematosus (SLE), type 1 diabetes (T1D), psoriasis, inflammatory bowel diseases (IBD), Chron's disease (CD), ulcerative colitis (UC), Graves disease, myesthenia gravis, scleroderma bullosa, Hashimoto's thyroiditis and ankylosing spondilitys.

The term “DC” as used herein refers to any member of a diverse population of morphologically similar cell types found in lymphoid and non-lymphoid tissues. These cells are characterized by their distinctive morphology and expression of MHC class II (Steinman et al, Ann. Rev. Immunol. 9:271-296). The term “DC” includes but is not limited to myeloid DC (expressing CD11c) comprising Langerhans cell, dermal and interstitial DC, and plasmacytoid DC (also called plasmacytoid monocytes or type I interferon-producing cells (IPC)) that are CD11c⁻/CD123⁺/CD4⁺ (see Fonteneau et al. 2003, Blood 101:3520-3526 and Vermi et al. 2003, J Pathology 200:255-268).

The term “allograft” as used herein cover all kinds of transplantation within the same species where donor tissue and recipient tissue differ in the expression of major and minor histocompatibility genes.

The term “treatment” and “treating” as used herein means the management and care of a patient for the purpose of combating a condition, such as a disease or a disorder. The term is intended to include the full spectrum of treatments for a given condition from which the patient is suffering, such as prevention of the condition, the delaying of the progression of the disease, disorder or condition, the alleviation or relief of symptoms and complications, and/or the cure or elimination of the disease, disorder or condition. The patient to be treated is preferably a mammal, in particular a human being.

The term “effective amount” as used herein means an amount that is sufficient to provide a clinical effect. It will depend on the means of administration, target site, state of the patient, whether the treatment takes place in the subject or on isolated cells, the frequency of treatment etc. Dosage ranges would ordinarily be expected from 0.1 microgram to 3000 microgram per kilogram of body weight per day. For a complete discussion of drug formulations and dosage ranges see Remington's Pharmaceutical Sciences, 18th Ed., (Mack Publishing Co., Easton, Pa., 1996).

It is to be understood that the present invention is not limited to the particular methodology, protocols and reagents described, as such may vary. It is also understood that the terminology used herein is for the purpose of describing particular embodiments only, and is not intended to limit the scope of the present invention.

Those of skill will readily appreciate that dose levels can vary as a function of the specific compound, the severity of the symptoms and the susceptibility of the subject to side effects. Preferred dosages for a given compound are readily determinable to those of skilled in the art by a variety of means. A preferred means is to measure the physiological potency of a given compound.

In the context of the present invention “administration”, “combined administration” or “combination therapy” refers to a treatment, management or prevention of autoimmune diseases or conditions and allograft rejection by administering IL-21 and any agent or combination of agents that interfere with the activation or persistence of autoreactive T and B cells and/or diminish the pathological response and modulates the disease. Said combination therapy can be performed by administering IL-21 prior to said agents or combination of agents and/or by simultaneous administration of IL-21 and said agents or combination of agents and/or by administration of IL-21 after administration of said agents or combination of agents.

In the context of the present invention the combinations provides an “effective amount” as applied to IL-21 or any of the combinations and refers to the amount of each component of the mixture which is effective for survival of the host.

### DESCRIPTION OF THE INVENTION

In one embodiment, the present invention relates to the use of IL-21, an analogue, a derivative or active fragment thereof, an IL-21 mimetic or an IL-21 polynucleotide for the preparation of a medicament for the treatment of diseases or conditions where T or B cells are involved.

In another embodiment, the present invention relates to the use of IL-21, an analogue, a derivative or active fragment thereof, an IL-21 mimetic or an IL-21 polynucleotide for the preparation of a medicament for the treatment of autoimmune diseases or conditions.

In another embodiment, the present invention relates to the use of IL-21, an analogue, a derivative or active fragment thereof, an IL-21 mimetic or an IL-21 polynucleotide for the preparation of a medicament for the treatment of RA.

In another embodiment, the present invention relates to the use of IL-21, an analogue, a derivative or active fragment thereof, an IL-21 mimetic or an IL-21 polynucleotide for the preparation of a medicament for the treatment of MS.

In another embodiment, the present invention relates to the use of IL-21, an analogue, a derivative or active fragment thereof, an IL-21 mimetic or an IL-21 polynucleotide for the preparation of a medicament for the treatment of T1D.

In another embodiment, the present invention relates to the use of IL-21, an analogue, a derivative or active fragment thereof, an IL-21 mimetic or an IL-21 polynucleotide for the preparation of a medicament for the treatment of alloresponse

In another embodiment, the present invention relates to the use of IL-21, an analogue, a derivative or active fragment thereof, an IL-21 mimetic or an IL-21 polynucleotide for the preparation of a medicament for prolonging allograft survival.

“IL-21” is described in International Patent Application publication no. WO 00/53761, published Sep. 14, 2000, which is hereby incorporated in this application in its entirety, discloses IL-21 (as “Zalpha11 ligand”) as SEQ ID No. 2, which is hereby incorporated in this application in its entirety, as well as methods for producing it and antibodies thereto and a polynucleotide sequence encoding IL-21 as SEQ ID No. 1 in the aforementioned application. The invention comprises their orthologs comprising at least 70%, at least 80%, at least 90%, at least 95%, or greater than 95% sequence identity. The present invention also includes the use of polypeptides that comprise an amino acid sequence having at least 70%, at least 80%, at least 90%, at least 95% or greater than 95% sequence identity to the sequence of amino acid residues 1 to 162, residues 41(Gln) to 148(Ile) of SEQ ID No: 2. Methods for determining percent identity are described below. The IL-21 polypeptides of the present invention have retained all or some of the biological activity of IL-21 which makes IL-21 useful for treating for example infections and cancer. Some of the polypeptides may also have a biological activity which is higher than the biological activity of IL-21.

The present invention embraces counterpart proteins and polynucleotides from other species (“orthologs”). Of particular interest are IL-21 polypeptides from other mammalian species, including rodent, porcine, ovine, bovine, canine, feline, equine, and other primates. Species orthologs of the human IL-21 protein can be cloned using information and compositions provided by the present invention in combination with conventional cloning techniques. As used and claimed, the language “an isolated polynucleotide which encodes a polypeptide, said polynucleotide being defined by SEQ ID NO:2 includes all allelic variants and species orthologs of this polypeptide.

The present invention also provides isolated protein polypeptides that are substantially identical to the protein polypeptide of SEQ ID NO: 2 and its species orthologs. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. The term “substantially identical” is used herein to denote polypeptides having 50%, preferably 60%, more preferably at least 80%, sequence identity to the sequence shown in SEQ ID NO:2 of WO00/53761 or species orthologs. Such polypeptides will more preferably be at least 90% identical, and most preferably 95% or more identical to SEQ ID NO:2, or its species orthologs. Percent sequence identity is determined by conventional methods. See, for example, Altschul et al., Bull. Math. Bio. 48: 603-616 (1986) and Henikoff and Henikoff, Proc. Natl. Acad. Sci. USA 89:1091510919 (1992). Sequence identity of polynucleotide molecules is determined by similar methods using a ratio as disclosed above.

Variant IL-21 polypeptides or substantially identical proteins and polypeptides are characterized as having one or more amino acid substitutions, deletions or additions. These changes are preferably of a minor nature, that is conservative amino acid substitutions (see Table 1) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A, Nilsson et al., EMBO J. 4:1075 (1985); Nilsson et al., Methods Enzymol. 198:3 (1991), glutathione S transferase, Smith and Johnson, Gene 67:31 (1988), or other antigenic epitope or binding domain. See, in general Ford et al., Protein Expression and Purification 2: 95-107 (1991). DNAs encoding affinity tags are available from commercial suppliers (e.g., Pharmacia Biotech, Piscataway, N.J.).

TABLE 1
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, Nmethylglycine, addo-threonine, methylthreonine, hydroxyethylcysteine, hydroxylethyl-homocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating nonnaturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Essential amino acids in the polypeptides of the present invention can be identified according to procedures known in the art, such as site-directed mutagenesis or alanine scanning mutagenesis [Cunningham and Wells, Science 244: 1081-1085 (1989)]; Bass et al., Proc. Natl. Acad. Sci. USA 88:4498-4502 (1991). In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand:protein interaction can also be determined by analysis of crystal structure as determined by such techniques as nuclear magnetic resonance, crystallography or photoaffinity labeling. See, for example, de Vos et ad., Science 255:306-312 (1992); Smith et al., J. Mod. Biod. 224:899-904 (1992); Wlodaver et ad., FEES Lett. 309:59-64 (1992). The identities of essential amino acids can also be inferred from analysis of homologies with related proteins.

Multiple amino acid substitutions can be made and tested using known methods of mutagenesis and screening, such as those disclosed by Reidhaar-Olson and Sauer, Science 241:53-57 (1988) or Bowie and Sauer Proc. Natl. Acad. Sci. USA 86:2152-2156 (1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837 (1991); Ladner et al., U.S. Pat. No. 5,223,409; Huse, WIPO Publication WO 92/06204) and region-directed mutagenesis, Derbyshire et al., Gene 46:145 (1986); Ner et al., DNA 7:127 (1988).

Mutagenesis methods as disclosed above can be combined with high-throughput screening methods to detect activity of cloned, mutagenized proteins in host cells. Preferred assays in this regard include cell proliferation assays and biosensor-based ligand-binding assays, which are described below. Mutagenized DNA molecules that encode active proteins or portions thereof (e.g., ligand-binding fragments) can be recovered from the host cells and rapidly sequenced using modern equipment. These methods allow the rapid determination of the importance of individual amino acid residues in a polypeptide of interest, and can be applied to polypeptides of unknown structure.

The present invention further provides a variety of other polypeptide fusions [and related multimeric proteins comprising one or more polypeptide fusions]. For example, a IL-21 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-IL-21 polypeptide fusions can be expressed in genetically engineered cells Auxiliary domains can be fused to IL-21 polypeptides to target them to specific cells, tissues, or macromolecules (e.g., collagen). For example, an IL-21 polypeptide or protein could be targeted to a predetermined cell type by fusing a polypeptide to a ligand that specifically binds to a receptor on the surface of the target cell. In this way, polypeptides and proteins can be targeted for therapeutic or diagnostic purposes. A IL-21 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9 (1996).

Derivatives of IL-21 comprises derivatisation or linking to another functional molecule. The linking can be chemical coupling, genetic fusion, non-covalent association or the like, to other molecular entities such as antibodies, toxins, radioisotope, cytotoxic or cytostatic agents.

Using the methods discussed above, one of ordinary skill in the art can prepare a variety of polypeptides that are substantially identical to SEQ ID NOs: 2 or allelic variants thereof, but which has the biological activity of IL-21. As expressed and claimed herein the language, “a polypeptide as defined by SEQ ID NO: 2” includes all allelic variants and species orthologs of the polypeptide.

The protein polypeptides of the present invention, including full-length proteins, protein fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed. (Cold Spring Harbor Laboratory Press, Cold Spring Harbor, N.Y., 1989), and Ausubel et al., ibid.

It is to be recognized that according to the present invention, when a cDNA is claimed as described above, it is understood that what is claimed are both the sense strand, the anti-sense strand, and the DNA as double-stranded having both the sense and antisense strand annealed together by their respective hydrogen bonds. Also claimed is the messenger RNA (mRNA) which encodes the polypeptides of the present invention, and which mRNA is encoded by the above-described cDNA. A messenger RNA (mRNA) will encode a polypeptide using the same codons as those defined above, with the exception that each thymine nucleotide (T) is replaced by a uracil nucleotide (U).

To direct an IL-21 polypeptide into the secretory pathway of a host cell, a secretory signal sequence (also known as a leader sequence, prepro sequence or pre sequence) is provided in the expression vector. The secretory signal sequence may be that of the protein, or may be derived from another secreted protein (e.g.,) or synthesized de novo. The secretory signal sequence is joined to the IL-21 DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

The invention also comprises chemical modifications of the IL-21 polypeptide. The chemical modification comprises covalent modifications with an organic agent capable of reacting with a selected side chain or a terminal residue. Examples of such modifications are wherein a lipophilic substituent is attached to one or more amino acid residues at a position relative to the amino acid sequence of SEQ ID NO:1 or 2 as described above. It is to be understood that an amino acid residues at the position relative to the amino acid sequence of SEQ ID NO:2 may be any amino acid residue and not only the amino acid residue naturally present at that position. In one embodiment the lipophilic substituent is attached to a lysine. One or more of the lysines in IL-21 could be derivatives as described in the application.

In other preferred embodiments, additional lysines are substituted, inserted into the sequence or added at the N-terminal or C-terminal, and then optionally derivatised.

Preferred regions of insertions are where the overall activity of the protein is not adversely affected. Preferred regions are the loop region. N-terminal and C-terminal truncations may occur simultaneously.

The term “lipophilic substituent” is characterised by comprising 4-40 carbon atoms and having a solubility in water at 20° C. in the range from about 0.1 mg/100 ml water to about 250 mg/100 ml water, such as in the range from about 0.3 mg/100 ml water to about 75 mg/100 ml water. For instance, octanoic acid (C8) has a solubility in water at 20° C. of 68 mg/100 ml, decanoic acid (C10) has a solubility in water at 20° C. of 15 mg/100 ml, and octadecanoic acid (C18) has a solubility in water at 20° C. of 0.3 mg/100 ml.

To obtain a satisfactory protracted profile of action of the IL-21 derivative, the lipophilic substituent attached to the IL-21 moiety, as an example comprises 4-40 carbon atoms, such as 8-25 carbon atoms. The lipophilic substituent may be attached to an amino group of the IL-21 moiety by means of a carboxyl group of the lipophilic substituent which forms an amide bond with an amino group of the amino acid to which it is attached. As an alternative, the lipophilic substituent may be attached to said amino acid in such a way that an amino group of the lipophilic substituent forms an amide bond with a carboxyl group of the amino acid. As a further option, the lipophililic substituent may be linked to the IL-21 moiety via an ester bond. Formally, the ester can be formed either by reaction between a carboxyl group of the IL-21 moiety and a hydroxyl group of the substituent-to-be or by reaction between a hydroxyl group of the IL-21 moiety and a carboxyl group of the substituent-to-be. As a further alternative, the lipophilic substituent can be an alkyl group which is introduced into a primary amino group of the IL-21 moiety.

In one embodiment of the invention the IL-21 derivative only has one lipophilic substituent attached to the IL-21 peptide.

In one embodiment of the invention the lipophilic substituent comprises from 4 to 40 carbon atoms.

In one embodiment of the invention the lipophilic substituent comprises from 8 to 25 carbon atoms.

In one embodiment of the invention the lipophilic substituent comprises from 12 to 20 carbon atoms.

In one embodiment of the invention the lipophilic substituent is attached to an amino acid residue in such a way that a carboxyl group of the lipophilic substituent forms an amide bond with an amino group of the amino acid residue.

In other preferred embodiments, additional lysines are substituted, inserted into the sequence or added at the N-terminal or C-terminal, and then optionally derivatised.

Preferred regions of insertions are where the overall activity of the protein is not adversely affected. Preferred regions are the loop region.

In one embodiment of the invention the lipophilic substituent is attached to an amino acid residue in such a way that an amino group of the lipophilic substituent forms an amide bond with a carboxyl group of the amino acid residue.

In one embodiment of the invention the lipophilic substituent is attached to the IL-21 peptide by means of a spacer.

In one embodiment of the invention the spacer is an unbranched alkane α,ω-dicarboxylic acid group having from 1 to 7 methylene groups, such as two methylene groups which spacer forms a bridge between an amino group of the IL-21 peptide and an amino group of the lipophilic substituent.

In one embodiment of the invention the spacer is an amino acid residue except a Cys residue, or a dipeptide. Examples of suitable spacers includes β-alanine, gamma-aminobutyric acid (GABA), γ-glutamic acid, succinic acid, Lys, Glu or Asp, or a dipeptide such as Gly-Lys. When the spacer is succinic acid, one carboxyl group thereof may form an amide bond with an amino group of the amino acid residue, and the other carboxyl group thereof may form an amide bond with an amino group of the lipophilic substituent. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the ε-amino group of Lys and the lipophilic substituent.

In one embodiment, such a further spacer is succinic acid which forms an amide bond with the ε-amino group of Lys and with an amino group present in the lipophilic substituent.

In another embodiment such a further spacer is Glu or Asp which forms an amide bond with the ε-amino group of Lys and another amide bond with a carboxyl group present in the lipophilic substituent, that is, the lipophilic substituent is a N^{ε}-acylated lysine residue.

In one embodiment of the invention the spacer is selected from the list consisting of β-alanine, gamma-aminobutyric acid (GABA), γ-glutamic acid, Lys, Asp, Glu, a dipeptide containing Asp, a dipeptide containing Glu, or a dipeptide containing Lys. In one embodiment of the invention the spacer is β-alanine. In one embodiment of the invention the spacer is gamma-aminobutyric acid (GABA). In one embodiment of the invention the spacer is γ-glutamic acid.

In one embodiment of the invention a carboxyl group of the parent IL-21 peptide forms an amide bond with an amino group of a spacer, and the carboxyl group of the amino acid or dipeptide spacer forms an amide bond with an amino group of the lipophilic substituent.

In one embodiment of the invention an amino group of the parent IL-21 peptide forms an amide bond with a carboxylic group of a spacer, and an amino group of the spacer forms an amide bond with a carboxyl group of the lipophilic substituent.

In one embodiment of the invention the lipophilic substituent comprises a partially or completely hydrogenated cyclopentanophenathrene skeleton.

In one embodiment of the invention the lipophilic substituent is an straight-chain or branched alkyl group.

In one embodiment of the invention the lipophilic substituent is the acyl group of a straight-chain or branched fatty acid.

In one embodiment of the invention the acyl group of a lipophilic substituent is selected from the group comprising CH₃(CH₂)ₙCO—, wherein n is 4 to 38, such as CH₃(CH₂)₆CO, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one embodiment of the invention the lipophilic substituent is an acyl group of a straight-chain or branched alkane α,ω-dicarboxylic acid.

In one embodiment of the invention the acyl group of the lipophilic substituent is selected from the group comprising HOOC(CH₂)ₘCO—, wherein m is 4 to 38, such as HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

In one embodiment of the invention the lipophilic substituent is a group of the formula CH₃(CH₂)ₚ((CH₂)_{q}COOH)CHNH—CO(CH₂)₂CO—, wherein p and q are integers and p+q is an integer of from 8 to 40, such as from 12 to 35.

In one embodiment of the invention the lipophilic substituent is a group of the formula CH₃(CH₂)ᵣCO—NHCH(COOH)(CH₂)₂CO—, wherein r is an integer of from 10 to 24.

In one embodiment of the invention the lipophilic substituent is a group of the formula CH₃(CH₂)ₛCO—NHCH((CH₂)₂COOH)CO—, wherein s is an integer of from 8 to 24.

In one embodiment of the invention the lipophilic substituent is a group of the formula COOH(CH₂)ₜCO— wherein t is an integer of from 8 to 24.

In one embodiment of the invention the lipophilic substituent is a group of the formula —NHCH(COOH)(CH₂)₄NH—CO(CH₂)ₙCH₃, wherein u is an integer of from 8 to 18.

In one embodiment of the invention the lipophilic substituent is a group of the formula —NHCH(COOH)(CH₂)₄NH—COCH((CH₂)₂COOH)NH—CO(CH₂)_{w}CH₃, wherein w is an integer of from 10 to 16.

In one embodiment of the invention the lipophilic substituent is a group of the formula —NHCH(COOH)(CH₂)₄NH—CO(CH₂)₂CH(COOH)NH—CO(CH₂)ₓCH₃, wherein x is an integer of from 10 to 16.

In one embodiment of the invention the lipophilic substituent is a group of the formula —NHCH(COOH)(CH₂)₄NH—CO(CH₂)₂CH(COOH)NHCO(CH₂)_{y}CH₃, wherein y is zero or an integer of from 1 to 22.

In one embodiment of the invention the lipophilic substituent is N-Lithocholoyl.

In one embodiment of the invention the lipophilic substituent is N-Choloyl.

In one embodiment of the invention the IL-21 derivative has one lipophilic substituent.

In one embodiment of the invention the IL-21 derivative has two lipophilic substituents.

In one embodiment of the invention the IL-21 derivative has three lipophilic substituents.

In one embodiment of the invention the IL-21 derivative has four lipophilic substituents.

The methods of the present invention also contemplate using chemically modified IL-21 compositions, in which a IL-21 polypeptide is linked with a polymer. Illustrative IL-21 polypeptides are soluble polypeptides that lack a functional transmembrane domain, such as a mature IL-21 polypeptide. Typically, the polymer is water soluble so that the IL-21 conjugate does not precipitate in an aqueous environment, such as a physiological environment. An example of a suitable polymer is one that has been modified to have a single reactive group, such as an active ester for acylation, or an aldehyde for alkylation, In this way, the degree of polymerization can be controlled. An example of a reactive aldehyde is polyethylene glycol propionaldehyde, or mono-(C1-C10) alkoxy, or aryloxy derivatives thereof (see, for example, Harris, et al., U.S. Pat. No. 5,252,714). The polymer may be branched or unbranched. Moreover, a mixture of polymers can be used to produce IL-21 conjugates.

IL-21 conjugates used for therapy can comprise pharmaceutically acceptable water-soluble polymer moieties. Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. An IL-21 conjugate can also comprise a mixture of such water-soluble polymers.

Percentage sequence identity between two amino acid sequences is determined by a Needleman-Wunsch alignment, useful for both protein and DNA alignments. For protein alignments the default scoring matrix used is BLOSUM50, and the penalty for the first residue in a gap is −12, while the penalty for additional residues in a gap is −2. The alignment may be made with the Align software from the FASTA package version v20u6 (W. R. Pearson and D. J. Lipman (1988), “Improved Tools for Biological Sequence Analysis”, PNAS 85:2444-2448; and W. R. Pearson (1990) “Rapid and Sensitive Sequence Comparison with FASTP and FASTA”, Methods in Enzymology, 183:63-98).

In one embodiment the polypeptide used in the present invention is an isolated polypeptide. In another embodiment the polynucleotide used in the present invention is an isolated polynucleotide.

It is preferred to purify the polypeptides of the present invention to: >80% purity, more preferably to >90% purity, even more preferably >95% purity with respect to contaminating macromolecules, particularly other proteins and nucleic acids, and free of infectious and pyrogenic agents, and particularly preferred is a pharmaceutically pure state, that is greater than 98% pure or preferable greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

In a further aspect of the invention the present IL-21, an analogue, a derivative or active fragment thereof, an IL-21 mimetic or an IL-21 polynucleotide are administered in combination with one or more active substances involved in autoimmune diseases or conditions in any suitable ratios.

In still a further aspect of the invention the present IL-21 peptide, an analogue, a derivative or active fragment thereof, an IL-21 mimetic or an IL-21 polynucleotide are administered alone or in combination with one or more active substances involved in allograft rejection in any suitable ratios.

The following of non-limiting examples of such active substances that can be used together with IL-21, an analogue, a derivative or active fragment thereof, an IL-21 mimetic or an IL-21 polynucleotide in combination therapy of autoimmune diseases or conditions and in allograft rejection is not intended in any way to limit the scope of the invention:
DC modifying agents
T cell modifying or suppressive agents
cytokines
growth factors
antigens that are known to be part of the pathogenesis in the relevant disease or condition
Cytokine antagonists
Cytokine receptor antagonists
Toll-like receptor (TLR) antagonists

Non-limiting examples of DC modifying agents are GM-CSF, Flt3-ligand, IL-10, TNF-α, viral IL-10, TGF-β, vitamin D receptors ligands, antagonists of CD40, antagonists of CD154, agonists of CD152, antagonists of IL-12, antagonists of IL-23, or antagonists of IFN-γ. Said agents may be administered simultaneous with IL-21, prior to IL-21 or after IL-21.

In one embodiment of the invention IL-21, an analogue or derivative thereof is administered in combination with one or more DC modifying agents.

In another embodiment of the invention IL-21, an analogue or derivative thereof is administered in combination with one or more of GM-CSF, IL-10, TNF-α, CD40 antagonist, CD154 antagonists.

In another embodiment of the invention IL-21, an analogue a derivative or active fragment thereof is combined with GM-CFS.

In another embodiment of the invention IL-21, an analogue a derivative or active fragment thereof is combined IL-10.

In another embodiment of the invention IL-21, an analogue a derivative or active fragment thereof is combined with TNF-α.

In another embodiment of the invention IL-21, an analogue a derivative or active fragment thereof is combined CD40 antagonist.

In another embodiment of the invention IL-21, an analogue a derivative or active fragment thereof is combined CD154 antagonist.

Non-limiting examples of T cell modifying and suppressing agents are IL-10, CTLA-4 agonist or CD3 antagonist.

In one embodiment of the invention IL-21, an analogue or derivative thereof is administered together with one or more T cell modifying and suppressing agents.

In another embodiment of the invention IL-21 analogue or derivative of IL-21 is combined with one or more of the compounds selected from the group comprising: IL-10, CTLA-4 agonist, CD3 antagonist.

In another embodiment of the invention IL-21, an analogue a derivative or active fragment thereof is combined CTLA-4 agonist.

In another embodiment of the invention IL-21, an analogue a derivative or active fragment thereof is combined with CD3 antagonist.

Non-limiting examples of cytokines are IL-10, TNF-α, TGF-β.

In one embodiment of the invention IL-21, an analogue a derivative or active fragment thereof is combined with one or more cytokines.

In another embodiment of the invention an IL-21 analogue or derivative of IL-21 is combined with one or more of the compounds selected from the group comprising: IL-10, TNF-α, TGF-β.

In another embodiment of the invention IL-21, an analogue a derivative or active fragment thereof is combined with TGF-β.

In one embodiment of the invention IL-21, an analogue a derivative or active fragment thereof is combined with one or more growth factors.

Antigens that are involved in the pathogenesis of autoimmune diseases may be administered prior to or simultaneous with IL-21, IL-21 mimetic or IL-21 polynucleotide with or without additional DC modifying agents and/or T cell modifying or suppressive agents.

Non-limiting examples of antigens that may be used in combination with IL-21 are collagen, myelin basic protein, myelin oligodendrocyte glycoprotein, proteolipid protein, insulin, glutamic acid decarboxylase, heat shock proteins and other autoantigens. The antigens may be conjugated to a DC targeting antibody such as for example DEC-205 specific antibody or other of the proteins mentioned above, in order to facilitate antigen delivery and processing by DC.

Other examples include fragments of above-mentioned antigens and peptides derived from autoantigens, including the above-mentioned antigens that can be presented on MHC class I or II molecules.

Other examples are lysates of cells or tissues expressing autoantigens including but not limited to pancreatic β-cells. In one embodiment, one or more antigens are administered in combination with IL-21, an IL-21 mimetic or an IL-21 polynucleotide with or without the administration of additional DC modifying agents and/or T cell modifying or suppressive agents for use in treating autoimmune diseases or conditions according to the present invention.

Non-limiting examples of cytokine antagonists are IL-2 antagonists, IL-6 antagonists, IL-12p40 antagonists, IL-12p70 antagonists and/or IL-23 antagonists.

In one embodiment of the invention IL-21, an analogue a derivative or active fragment thereof is combined with one or more cytokine antagonists.

In another embodiment of the invention an IL-21 analogue or derivative of IL-21 is combined with one or more of the compounds selected from the group comprising: IL-2 antagonists, IL-6 antagonists, IL-12p40 antagonists, IL-12p70 antagonists, IL-23 antagonists.

In another embodiment of the invention IL-21, an analogue a derivative or active fragment thereof is combined with IL-2 antagonists.

In another embodiment of the invention IL-21, an analogue a derivative or active fragment thereof is combined with IL-6 antagonists.

In another embodiment of the invention IL-21, an analogue a derivative or active fragment thereof is combined with IL-12p40 antagonists.

In another embodiment of the invention IL-21, an analogue a derivative or active fragment thereof is combined with IL-12p70 antagonists.

In another embodiment of the invention IL-21, an analogue a derivative or active fragment thereof is combined with IL-23 antagonists.

Non-limiting examples of cytokine receptor antagonists are CD25 antagonists, CD122 antagonists, IL-6R antagonists, IL-12R antagonists and/or IL-23R antagonists.

In one embodiment of the invention IL-21, an analogue a derivative or active fragment thereof is combined with one or more cytokine receptor antagonists.

In another embodiment of the invention an IL-21 analogue or derivative of IL-21 is combined with one or more of the compounds selected from the group comprising: CD25 antagonists, CD122 antagonists, IL-6R antagonists, IL-12R antagonists and/or IL-23R antagonists.

In another embodiment of the invention IL-21, an analogue a derivative or active fragment thereof is combined with CD25 antagonists.

In another embodiment of the invention IL-21, an analogue a derivative or active fragment thereof is combined with CD122 antagonists.

In another embodiment of the invention IL-21, an analogue a derivative or active fragment thereof is combined with IL-6R antagonists.

In another embodiment of the invention IL-21, an analogue a derivative or active fragment thereof is combined with IL-12R antagonists.

In another embodiment of the invention IL-21, an analogue a derivative or active fragment thereof is combined with IL-23R antagonists.

In one embodiment of the invention IL-21, an analogue a derivative or active fragment thereof is combined with one or more Toll-like receptor (TLR) antagonists.

A conjugate of IL-21 and a monoclonal antibody (mAb) or a conjugate of IL-21 and a mAb fragment (e.g. Fab or F(ab′)₂ fragments) may be used to target IL-21 to the DC or a subset of DC.

Non-limiting examples of surface molecules expressed by DC or DC subsets that might be targeted by an IL-21 mAb conjugate are CD11c, DEC-205, CD123 (IL-3Rα), BDCA-2, BDCA-3, BDCA-4, CD206 (mannose receptor), CD207 (Langerin), CD208 (DC-LAMP), CD209 (DC-SIGN) and CLA/HECA. In one embodiment IL-21 conjugated to a DC-binding mAb, Fab fragment or F(ab′)₂ fragment of the mAb is administered for use in treating autoimmune diseases or conditions or allograft rejection according to the present invention. An IL-21 mAb conjugate as described above may be used in combination with other DC modifying agents and/or T cell modifying or suppressive agents and/or antigens as described above.

DC isolated from the subjects may be cultured together with IL-21 or an IL-21 mimetic in vitro to induce a regulatory or tolerogenic phenotype. Furthermore, additional DC modifying agents as described above may be added to enhance the development, differentiation and proliferation of regulatory or tolerogenic DC. Furthermore, antigens, fragments thereof, peptides derived from autoantigens, and cell or tissue lysates may be added to the culture to allow presentation of relevant antigens on MHC (HLA) molecules expressed by the DC. Following in vitro culture the DC may be reintroduced in vivo to promote suppression of auto- and alloreactivity. In one embodiment, DC are isolated from the subject suffering from an autoimmune disease or condition and treated in vitro with IL-21, or an IL-21 mimetic with or without the administration of additional DC modifying agents and/or antigens as described above, followed by reintroduction in vivo. Subsequent treatment of a DC expanding or modifying agent in vivo may be used to further the beneficial response. In one embodiment, DC are isolated from the donor of the allograft and treated in vitro with IL-21, or an IL-21 mimetic with or without the administration of additional DC modifying agents as described above, followed by introduction in vivo in the recipient of the allograft. Subsequent treatment of a DC expanding or modifying agent in vivo may be used to further the beneficial response. Such agents include but are not limited to the DC modifying agents mentioned above.

### Pharmaceutical Compositions

IL-21 or other IL-21 polypeptides for use in treating autoimmune diseases or conditions or allograft rejection according to the present invention may be administered alone or in combination with pharmaceutically acceptable carriers or excipients, in either single or multiple doses. The formulation of the combination may be as one dose unit combining the compounds, or they may be formulated as separate doses. The pharmaceutical compositions comprising IL-21 or other IL-21 polypeptides for use in treating autoimmune diseases or conditions or allograft rejection according to the present invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The compositions may appear in conventional forms, for example capsules, tablets, aerosols, solutions or suspensions.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen. The route of administration may be any route, which effectively transports the active compound to the appropriate or desired site of action.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, crèmes, gels, inhalants, dermal patches, implants etc.

A typical oral dosage is in the range of from about 0.001 to about 100 mg/kg body weight per day, such as from about 0.01 to about 50 mg/kg body weight per day, for example from about 0.05 to about 10 mg/kg body weight per day administered in one or more dosages such as 1 to 3 dosages. The exact dosage will depend upon the nature of the IL-21 polypeptide chosen, the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, for example from about 0.1 to about 500 mg, such as from about 0.5 mg to about 200 mg.

For parenteral routes such as intravenous, intrathecal, intramuscular and similar administration, typically doses are in the order of about half the dose employed for oral administration.

Non-protein IL-21 mimetics for use in treating autoimmune diseases or conditions or allograft rejection according to the present invention are generally utilized as the free substance or as a pharmaceutically acceptable salt thereof. Examples are an acid addition salt of a compound having the utility of a free base and a base addition salt of a compound having the utility of a free acid. The term “pharmaceutically acceptable salts” refers to non-toxic salts of such compounds which are generally prepared by reacting the free base with a suitable organic or inorganic acid or by reacting the acid with a suitable organic or inorganic base. When such a compound contains a free base such salts are prepared in a conventional manner by treating a solution or suspension of the compound with a chemical equivalent of a pharmaceutically acceptable acid. When such a compound contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds of the invention and these form a further aspect of the invention.

Salts of IL-21 polypeptides are especially relevant when the protein is in solid or crystalline form

For parenteral administration, solutions of the IL-21 polypeptides or IL-21 mimetics in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining a IL-21 polypeptide or IL-21 mimetic for use in treating autoimmune diseases or conditions or allograft rejection according to the present invention and the pharmaceutically acceptable carriers are then readily administered in a variety of dosage forms suitable for the disclosed routes of administration. The formulations may conveniently be presented in unit dosage form by methods known in the art of pharmacy.

For nasal administration, the preparation may contain a IL-21 polypeptide or IL-21 mimetic dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Formulations of IL-21 polypeptides or IL-21 mimetics, optionally together with the combination agent for use in treating autoimmune diseases or conditions or allograft rejection according to the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the IL-21 polypeptides or IL-21 mimetics, optionally together with the combination agent in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions of IL-21 polypeptides or IL-21 mimetics, optionally together with the combination agent for use in treating autoimmune diseases or conditions or allograft rejection according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservatives and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The IL-21 polypeptides or IL-21 mimetics, optionally together with the combination agent for use in treating autoimmune diseases or conditions or allograft rejection according to the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the IL-21 polypeptides or IL-21 mimetics for use in treating autoimmune diseases or conditions or allograft rejection according to the present invention may form solvates with water or common organic solvents. Such solvates are also encompassed within the scope of the invention.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

The IL-21 polypeptides or IL-21 mimetics, optionally together with the combination agent for use in treating autoimmune diseases or conditions or allograft rejection according to the present invention may be administered to a mammal, especially a human, in need of such treatment. Such mammals include also animals, both domestic animals, e.g. household pets, and non-domestic animals such as wildlife.

Pharmaceutical compositions containing a compound according to the invention may be administered one or more times per day or week, conveniently administered at mealtimes. An effective amount of such a pharmaceutical composition is the amount that provides a clinically significant effect. Such amounts will depend, in part, on the particular condition to be treated, age, weight, and general health of the patient, and other factors evident to those skilled in the art.

### EXAMPLES

TABLE A

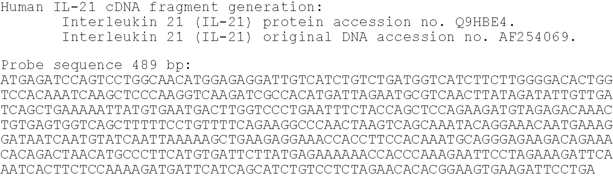

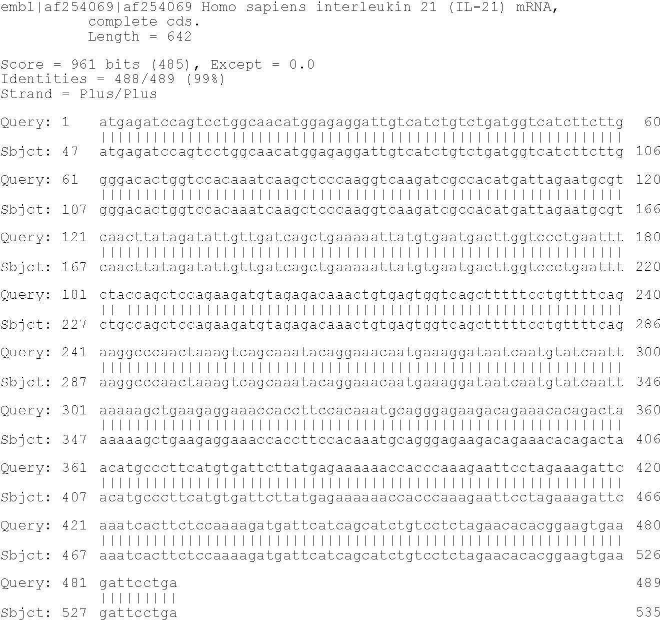

TABLE B

Human IL-21 amino acid sequence protein accession no.
Q9HBE4, also shown as SEQ ID No. 2, including the signal
peptide comprising residues 1 to 29:

1...MRSSPGNMERIVICLMVIFLGTLVHKSSSQGQDRHMIRMRQLIDIVDQLK...50
51..NYVNDLVPEFLPAPEDVETNCEWSAFSCFQKAQLKSANTGNNERIINVSI...100
101.KKLKRKPPSTNAGRRQKHRLTCPSCDSYEKKPPKEFLERFKSLLQKMIHQ...150
151 HLSSRTHGSEDS

### Pharmacological Methods

A method to demonstrate that DC cultured with IL-21 and other DC modifying agents and/or antigens can be used to induce regulatory T cells: DC cultures are prepared from peripheral blood monocytes isolated from healthy donors as described by Gilliet et al. (J. Exp. Med. 2002, 195:695-704). IL-21 and/or other DC modifying agents may be added during the first 5 days of culture or during the following 24 h stimulation period, and simulation with CD40L-transfected cells may be omitted. Naïve CD8⁺ T cells are prepared and cultured together with the DC in mixed leukocyte reactions, proliferation assays, cytotoxicity assay, suppression assays, coculture experiments and cytokine measurement as described by Gilliet et al. (J. Exp. Med. 2002, 195:695-704). Alternatively, DC are prepared as described by Sato et al. (Blood 2003, 101: 3581-9). IL-21 and/or other DC modifying agents may be added during the first 7 days of culture or during the following 3 day stimulation period. Preparation of regulatory T cells by coculture with the DC and analysis of regulatory T cell function is carried out in mixed leukocyte reactions, cytotoxicity assay, suppression assays and coculture experiments as described by Sato et al. (Blood 2003, 101: 3581-9). Alternatively, DC are isolated from the spleens of mice as described by O'Connel et al. (J. Imm. 2002, 168:143-154) or from bone marrow-derived DC are isolated and cultured as described by Haase et al. (Immunology 2002, 107:489-499). IL-21 and/or other DC modifying agents may be added during the initial culture or during a subsequent 24 h stimulation period. The DC are subsequently tested for their ability to induce regulatory T cells in proliferation assays, mixed leukocyte reactions and by measurement of cytokine release as described by O'Connel et al. (J. Imm. 2002, 168:143-154) or Feili-Hariri et al. (Eur. J. Immunol. 2002, 32:2021-2030).

A method to demonstrate that in vivo treatment with IL-21 with or without other DC modifying agents and/or immunosuppressive agents can be used to prolong allograft survival: Mice are treated with IL-21 and/or other DC modifying agents for up to two weeks and rendered diabetic by a single injection of streptozotocin. At least 250 allogeneic islets of Langerhans are engrafted under the kidney capsule, and the mice are continuously treated with IL-21 and/or other DC modifying agents and/or immunosuppressive agents. Graft survival is measured by measurement of the blood glucose level and by immunohistochemistry: a non-fasting blood glucose above 20 mM indicates graft failure. A similar method is described by Adorini et al. (J. Cell. Biochem. 2003, 88:227-33).

A method to demonstrate that DC cultured with IL-21 and other DC modifying agents and immunosuppressive agents can be used to prolong allograft survival: Mouse splenic or bone marrow-derived DC are isolated as described above (O'Connel et al., J. Imm. 2002, 168:143-154 and Haase et al., Immunology 2002, 107:489-499) and IL-21 and/or other DC modifying agents may be added during the initial culture or during a subsequent 24 h stimulation period. The DC's are subsequently injected into allogeneic hosts and the ability to protect against allograft rejection is tested by transplantation of allogeneic islets (autologous to the DC) as described above or by allogeneic heart transplantation as described by O'Connel et al. (J. Imm. 2002, 168:143-154).

A method to demonstrate that DC cultured with IL-21 and other DC modifying agents and/or antigens can be used to treat RA: Mouse splenic or bone marrow-derived DC are isolated as described above (O'Connel et al., J. Imm. 2002, 168:143-154 and Haase et al., Immunology 2002, 107:489-499). IL-21 and/or other DC modifying agents may be added during the initial culture or during a subsequent 24 h stimulation period. Arthritis is induced by collagen injection and treated with autologous DC as described by Morita et al. (J. Clin. Invest. 2001, 107:1275-1284). The effect of the treatment is evaluated by the incidence, mean percentages of arthritic limbs, and mean clinical score of CIA and the anti-collagen II antibody titer as described by Morita et al. (J. Clin. Invest. 2001, 107:1275-1284).

A method to demonstrate that in vivo treatment with IL-21 with or without other DC modifying agents or T cell modifying or suppressive agents and/or antigens can be used to treat RA: Arthritis is induced by collagen injection and treated with autologous DC as described by Morita et al. (J. Clin. Invest. 2001, 107:1275-1284). The mice are injected with IL-21 daily or every other day together with or without other DC modifying agents or T cell modifying or suppressive agents, for example a TNF-α antagonist. Treatment with IL-21 may be initiated both before and after the onset of clinical symptoms. The effect of the treatment is evaluated by the incidence, mean percentages of arthritic limbs, and mean clinical score of CIA and the anti-collagen II antibody titer as described by Morita et al. (J. Clin. Invest. 2001, 107:1275-1284).

A method to demonstrate that DC cultured with IL-21 and other DC modifying agents and/or antigens can be used to treat MS. Mouse splenic or bone marrow-derived DC are isolated as described above (O'Connel et al., J. Imm. 2002, 168:143-154 and Haase et al., Immunology 2002, 107:489-499). IL-21 and/or other DC modifying agents may be added during the initial culture or during a subsequent 4-24 h stimulation period. Furthermore, the DC may be loaded with MOG peptide, MBP peptide or PLP peptide during a subsequent 4-24 h stimulation period. Experimental autoimmune encephalomyelitis (EAE) is induced in my by immunization with MOG peptide, MBP peptide or PLP peptide in Complete Freunds Adjuvant followed by injection of pertussis toxin day 0 and 2 as described by Menges et al. (J. Exp. Med. 2002, 195:15-21). DC are injected once or repeatedly at later timepoints and the disease score and cytokine production is evaluated as described by Menges et al. (J. Exp. Med. 2002, 195:15-21).

A method to demonstrate that in vivo treatment with IL-21 with or without other DC modifying agents or T cell modifying or suppressive agents and/or antigens can be used to treat MS: Experimental autoimmune encephalomyelitis (EAE) is induced in my by immunization with MOG peptide, MBP peptide or PLP peptide in Complete Freunds Adjuvant followed by injection of pertussis toxin day 0 and 2 as described by Menges et al. (J. Exp. Med. 2002, 195:15-21). The mice are injected with IL-21 daily or every other day together with or without other DC modifying agents and/or antigens involved in the pathogenesis of EAE, for example MOG, MBP or PLP or peptides derived from these proteins. Treatment with IL-21 may be initiated both before and after the onset of clinical symptoms. DC are injected once or repeatedly at later time points and the disease score and cytokine production is evaluated as described by Menges et al. (J. Exp. Med. 2002, 195:15-21).

A method to demonstrate that DC cultured with IL-21 and other DC modifying agents and/or antigens can be used to treat T1D: Splenic or bone marrow-derived DC from pre-diabetic non-obese diabetic (NOD) mice are isolated as described above (O'Connel et al., J. Imm. 2002, 168:143-154 and Haase et al., Immunology 2002, 107:489-499). IL-21 and/or other DC modifying agents may be added during the initial culture or during a subsequent 4-24 h stimulation period. Furthermore, the DC may be loaded with insulin, GAD65 or HSP60 peptides during a subsequent 4-24 h stimulation period. DC are injected once or repeatedly injected into pre-diabetic NOD mice as described by Feili-Hariri et al. (Eur. J. Immunol. 2002, 32:2021-2030), and the development of diabetes is followed by measurement of the blood glucose level.

A method to demonstrate that in vivo treatment with IL-21 with or without other DC modifying agents or T cell modifying or suppressive agents and/or antigens can be used to treat T1D: NOD mice, 4-12 weeks of age are injected with 1 L-21 daily or every other day together with or without other DC modifying agents and/or antigens involved in the pathogenesis of diabetes, for example insulin, GAD65 or HSP60 or peptides derived from these proteins. The development of diabetes is followed by measurement of the blood glucose level.

A method to demonstrate that treatment with IL-21 conjugated to a DC targeting antibody preferentially induce signaling in DC: Mice are injected once with IL-21 conjugated to a DC targeting antibody or placebo, and blood samples are drawn at selected time points hereafter (15 min to 4 hrs) and analyzed for Stat1, Stat3 and Stat5 phosphorylation by staining with Stat1, Stat3 and Stat5 specific antibodies together with antibodies that can identify the leukocyte subset, followed by flow cytometric analysis. These proteins are known to be phosphorylated after engagement of IL-21R.

1. A method of treating a T cell-mediated or B-cell mediated disease or condition, in a subject in need thereof, comprising administering an effective amount of (a) interleukin-21 (“IL-21”), (b) an IL-21 analogue, (c) a derivative of IL-21 or an IL-21 analogue, or (d) an active fragment of IL-21 or an IL-21 analogue, (e) an IL-21 mimetic, or (f) an IL-21 polynucleotide to the subject so as to treat the disease or condition.

2. The method of claim 1, wherein the method comprises administering an effective amount of an IL-21 polypeptide comprising an amino acid sequence having at least 80% sequence identity to residues 30-162 of SEQ ID NO: 2.

3. The method of claim 2, wherein the IL-21 polypeptide comprises an amino acid sequence having at least 95% identity to residues 30-162 of SEQ ID NO:2.

4. The method of claim 2, wherein the IL-21 polypeptide has at least about 80% identity to SEQ ID NO:2.

5. The method of claim 4, wherein the IL-21 polypeptide is IL-21 or a derivative of IL-21.

6. The method of claim 1, wherein the disease or condition is an autoimmune disease.

7. The method of claim 6, wherein the autoimmune disease is rheumatoid arthritis (“RA”).

8. The method of claim 6, wherein the autoimmune disease is multiple sclerosis (“MS”).

9. The method of claim 6, wherein the autoimmune disease is type 1 diabetes (“T1D”).

10. The method of claim 1, wherein the disease or condition is allograft rejection.

11. The method of claim 1, wherein the method comprises administering at least one second agent that is useful in the treatment of the disease or condition.

12. The method of claim 11, wherein the second agent is a DC modifying agent.

13. The method of claim 11, wherein the second agent is a T cell modifying or T cell suppressive agent.

14. The method of claim 11, wherein the second agent is a cytokine.

15. The method of claim 11, wherein the second agent is a growth factor.

16. The method of claim 11, wherein the second agent is collagen, myelin basic protein, myelin oligodendrocyte glycoprotein, proteolipid protein, insulin, glutamic acid decarboxylase, or a heat shock protein.

17. The method of claim 11, wherein the second agent is a cytokine antagonist.

18. The method of claim 11, wherein the second agent is a cytokine receptor antagonist.

19. The method of claim 11, wherein the second agent is a Toll-like receptor (TLR) antagonist.

20. The method of claim 11, wherein the second agent is a disease-specific antigen.
